(19) United States
(12) Patent Application Publication
(10) Pub. No.: US 2026/0167155 A1
(43) Pub. Date: Jun. 18, 2026

(54) ULTRAVIOLET LAMP DEVICE FOR VEHICLE

(71) Applicants: HYUNDAI MOTOR COMPANY, Seoul (KR); KIA CORPORATION, Seoul (KR); Applicom, Seoul (KR); KBI DONGKOOK IND. CO., LTD., Gyeongju-si (KR)

(72) Inventors: Han-Joo JANG, Hwaseong-si (KR); Chang-Woo KANG, Asan-si (KR); Sung-Jun AN, Seongnam-si (KR)

(21) Appl. No.: 19/281,241

(22) Filed: Jul. 25, 2025

(30) Foreign Application Priority Data
Dec. 18, 2024 (KR) 10-2024-0189746

(51) Int. Cl. B60S 1/64; A61L 2/10; B60Q 3/68; B60Q 3/70

(52) U.S. Cl. CPC B60S 1/64; A61L 2/10; B60Q 3/68; B60Q 3/70; A61L 2103/75; A61L 2202/11; A61L 2202/121; B60Q 2500/00

(57) ABSTRACT

An ultraviolet (UV) lamp device for a vehicle includes a housing, a cover that covers a surface of the housing, a lamp disposed in the housing and configured to emit UV light, and a pair of electrodes configured to support the lamp, spaced apart from each other, and configured to apply a voltage to the lamp. The electrodes define a groove that seats the lamp and supports portions of a bottom surface and a side surface of the lamp.

### CROSS-REFERENCES TO RELATED APPLICATIONS

This application claims priority to Korean Patent Application No. 10-2024-0189746, filed on Dec. 18, 2024, which is incorporated herein by reference in its entirety.

### TECHNICAL FIELD

The present disclosure relates to an ultraviolet (UV) lamp device that is compact for a vehicle in consideration of efficiency.

### BACKGROUND

Ultraviolet (UV) sterilization devices generate UV light to sterilize bacteria and fungi. For example, the UV sterilization devices may include a UV lamp device that generates UV light using a UV lamp.

The UV lamp generates UV light of various wavelengths according to materials provided inside the UV lamp. For example, UV light of various wavelengths may include UV-A (400 nm to 315 nm), UV-B (315 nm to 280 nm), and UV-C (280 nm to 100 nm). When bacteria and fungi are irradiated with ultraviolet light, their deoxyribonucleic acids (DNAs) are damaged, and they die. That is, UV light may have an effective sterilization effect on various bacteria by damaging DNAs of living organisms. Among various ultraviolet wavelengths, UV light of UV-C wavelength may be suitable for sterilization.

UV sterilization may be applied to closed vehicle interiors. However, conventional ultraviolet sterilization devices may not be suitable for sterilizing vehicle interiors due to their size.

In some cases, conventional lamps may not output UV light, especially UV-C, with sufficient intensity due to their structural characteristics. In some cases, due to the nature of the lamp, light may be output in all directions from the lamp. In some cases, where the lamp is disposed in a structure for placing the lamp, the light emitted in a direction of a contact with the structure is absorbed or blocked by the structure, resulting in a decrease of light output.

### SUMMARY

The present disclosure describes an ultraviolet (UV) lamp device that can structurally exhibit sufficient efficiency in a narrow vehicle interior.

According to one aspect of the subject matter described in this application, an ultraviolet (UV) lamp device for a vehicle includes a housing, a cover that covers a surface of the housing, a lamp disposed in the housing and configured to emit UV light, and a pair of electrodes configured to apply a voltage to the lamp, the pair of electrodes being spaced apart from each other and supporting the lamp, where the pair of electrodes define a groove that accommodates at least a portion of the lamp and supports a bottom surface of the lamp and a side surface of the lamp.

Implementations according to this aspect can include one or more of the following features. For example, a depth of a lower end portion of the groove can be less than or equal to a radius of the lamp, where an inner lower surface of the lower end portion of the groove has a semi-cylindrical outer surface corresponding to a shape of the lamp.

In some examples, an upper end portion of the groove can extend upward from the lower end portion of the groove and be inclined with respect to a bottom surface of the pair of electrodes. In some examples, a depth of the groove can be less than or equal to a diameter of the lamp. In some implementations, an inclination angle of the upper end portion of the groove can range from 30° to 60° with respect to the bottom surface of the pair of electrodes. For instance, the inclination angle of the upper end portion of the groove can be 45°±5° with respect to the bottom surface of the pair of electrodes.

In some implementations, the UV lamp device can include a trigger disposed adjacent to the lamp and configured to cause the lamp to emit the UV light. In some examples, the trigger can include a cap and a coil disposed inside the cap. In some examples, the pair of electrodes can define trigger grooves that receive end portions of the trigger, respectively.

In some implementations, the UV lamp device can include a filter coupled to an open surface of the housing and configured to transmit a predetermined wavelength range of light.

In some examples, the cover can define a pair of through-holes, and the pair of electrodes can include terminals that pass through the pair of through-holes and are exposed to an outside of the cover.

In some implementations, the UV lamp device can include a reflector disposed between the pair of electrodes and configured to support the lamp. For example, the reflector can be in contact with the pair of electrodes. In some examples, the reflector can define a groove that has a cross-sectional shape corresponding to the semi-cylindrical outer surface of the groove of the pair of electrodes.

In some implementations, the UV lamp device can include a reflector that includes a first body disposed between a first electrode of the pair of electrodes and the housing, a second body disposed between a second electrode of the pair of electrodes and the housing, and a third body disposed between the pair of electrodes. In some examples, the reflector can further include an upper surface portion that connects between upper surfaces of the first body and the third body and that connects between an upper surface of the second body and the upper surface of the third body.

In some examples, the first body and the second body can define passing-through portions through which the lamp passes, and the third body can define a groove that has a cross-sectional shape corresponding to the semi-cylindrical outer surface of the groove of the pair of electrodes. In some examples, the third body has a first side and a second side with respect to the groove of the third body, where the second side is stepped relative to the first side, and the UV lamp device further includes a trigger disposed at the second side of the third body and configured to cause the lamp to emit the UV light.

In some implementations, the UV lamp device can include a pressurizing part that extends from at least one of the first body or the second body in a longitudinal direction of the lamp and is configured to be pressed by the cover. For examples, the pressurizing part can include a pair of pressurizing supports that extend from a side surface of the first body or the second body in the longitudinal direction, each of the pair of pressurizing supports having a notch defined at an upper portion thereof, and a pair of deformation portions that extend from upper ends of the pair of the pressurizing supports, respectively, and face each other, the pair of deformation portions being configured to be pressed by the cover.

### BRIEF DESCRIPTION OF THE DRAWINGS

FIG. 1 is a front perspective view illustrating an example of an ultraviolet (UV) lamp device.

FIG. 2 is a rear perspective view of the UV lamp device of FIG. 1.

FIGS. 3 and 4 are diagrams illustrating the UV lamp device of FIG. 1 that is partially open.

FIG. 5 is a diagram illustrating an operation of a lamp in related art.

FIG. 6 is a diagram illustrating an operation of a lamp in the UV lamp device according to the present disclosure.

FIG. 7 is a diagram illustrating an output result of the UV lamp device.

FIGS. 8 and 9 are diagrams illustrating example output data of the UV lamp device.

FIG. 10 is a diagram illustrating an example configuration of the UV lamp.

FIG. 11 is a diagram illustrating a side cross-sectional shape of the UV lamp of FIG. 10.

FIG. 12 is a front open perspective view illustrating an example of a UV lamp device.

FIG. 13 is a rear open perspective view of the UV lamp device of FIG. 12.

FIG. 14 is a front perspective view illustrating a reflector of the UV lamp device of FIG. 12.

FIG. 15 is a rear perspective view illustrating the reflector.

FIGS. 16 and 17 are diagrams illustrating example output data of the UV lamp device of FIG. 12.

FIG. 18 is a diagram illustrating an example configuration of a reflector in a UV lamp device.

FIG. 19 is a diagram illustrating a side cross-sectional shape of the reflector of FIG. 18.

FIG. 20 is a diagram illustrating an example configuration of a UV lamp device.

FIGS. 21 and 22 are flowcharts illustrating example methods of monitoring light output from a UV lamp device.

### DETAILED DESCRIPTION

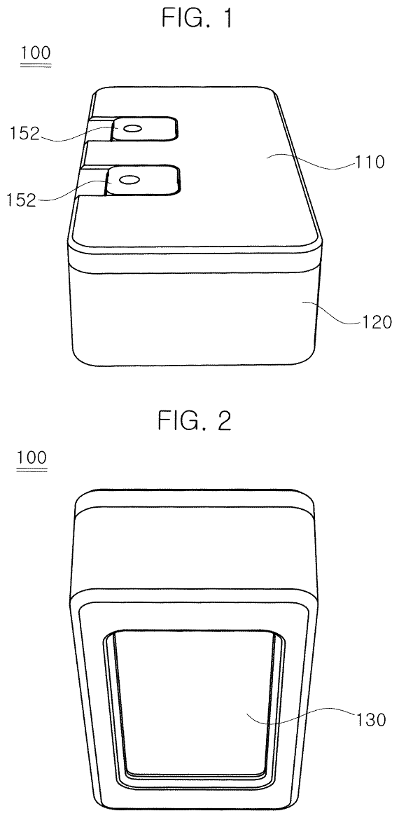

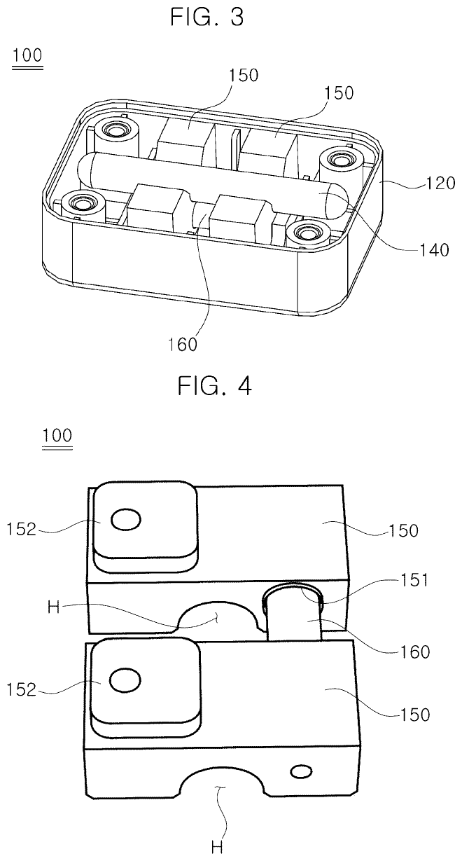

FIG. 1 is a front perspective view illustrating an example of an ultraviolet (UV) lamp device, and FIG. 2 is a rear perspective view. FIGS. 3 and 4 are diagrams illustrating a state in which the UV lamp device is partially open.

Referring to FIGS. 1 to 4, in some implementations, an ultraviolet (UV) lamp device 100 includes a cover 110, a housing 120, a filter 130, a lamp 140, an electrode 150, a trigger 160, and a controller. In some examples, the UV lamp device 100 can further include a lamp fixing part.

The UV lamp device 100 outputs light with a UV wavelength range, particularly, light with a UV-C wavelength range. In this case, the UV lamp device 100 significantly improves light efficiency of a UV wavelength range to be output by utilizing structural characteristics even at the same output.

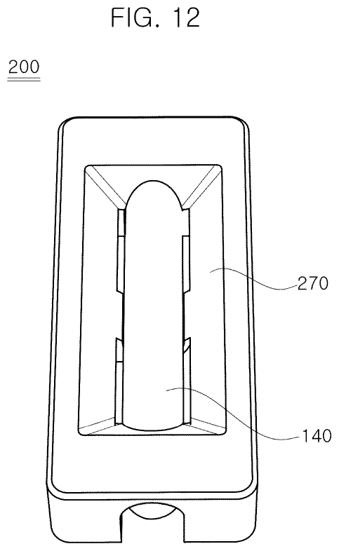

The cover 110 is coupled to the housing 120 to prevent the remaining components of the UV lamp device 100 within the housing 120 from being separated out of the UV lamp device 100. In addition, the cover 110 pressurizes a reflector according to a structure of the reflector (described below with reference to FIG. 12) inside the UV lamp device 100 to stably fix the lamp 140 inside the reflector.

In some examples, as shown in FIG. 1, the cover 110 has a structure in which through-holes are formed and terminals 152 of two electrodes 150, which will be described below, are exposed to the outside through the through-holes. Thus, power is supplied from the outside through the two electrodes 150, allowing the two electrodes 150 to supply voltage to the lamp 140.

The housing 120 provides a space in which the remaining components inside the UV lamp device 100 to be disposed and fixed. An interior of the housing 120 is implemented to correspond to a maximum length and a maximum width of the component inside the UV lamp device 100 so that the remaining components inside the UV lamp device 100 disposed in the housing 120 can be disposed and fixed.

In some examples, as shown in FIG. 2, the housing 120 is implemented in a form in which one surface or a portion within one surface is open so that light emitted from the lamp 140 can be output to the outside.

The filter 130 is mounted in an open portion inside the housing 120 to separate the outside from the interior of the housing 120 and, simultaneously, light with a wavelength range out of a predetermined wavelength range is filtered. For example, the UV lamp device 100 can be a device for precisely outputting only a UV-C wavelength range among the UV wavelength range. In some cases, even when light with the UV wavelength range is output from the lamp 140, there is a probability possibility that only the UV-C wavelength range is not output precisely, and there can be cases in which only UV light with a specific wavelength range (for example, around 230 nm) of the UV-C wavelength range should be output. In some implementations, the filter 130 is mounted in the open portion inside the housing 120. The filter 130 transmits only light with a predetermined wavelength range and filters light with the remaining wavelength range. Thus, light with a uniform UV wavelength range (especially, UV-C) can be output from the UV lamp device 100.

The lamp 140 outputs light with the UV wavelength range, specifically, the UV-C wavelength range. The lamp 140 receives a high voltage from the electrodes 150. The lamp 140 includes a gas component (e.g., mercury, etc.) that emits light with the UV wavelength range when discharged, and when the lamp 140 is triggered by the trigger 160, the lamp 140 receives a high voltage from the electrodes 150 to discharge light with the UV wavelength range. The trigger 160 is configured to form initial plasma or increase a plasma discharge rate. The lamp 140 operates in the above-described manner and can be implemented as, for example, an excimer lamp.

The electrodes 150 support the lamp 140, apply voltages to the lamp 140, and reflect light output from the lamp 140.

As shown in the drawing, the electrode 150 has a u-shaped groove in a direction in which the electrode 150 is mounted within the housing 120. That is, by forming a groove H in which the lamp 140 can be settled, the electrode 150 is formed to support and surround not only a bottom surface of the lamp 140 inserted into the groove H but also a portion of a side surface. In some examples, a depth of the groove H corresponds to a diameter of the lamp 140 or is the diameter or less.

Through this structure, the electrode 150 reflects light incident on the electrode 150. The electrode 150 can be implemented with a component that has electrical conductivity, excellent reflection characteristics (a preset reference value or more), and an excellent light flux maintenance rate (a preset reference value or more) of the UV wavelength range (especially, a UV-C wavelength range). As a representative example, the electrode 150 can be implemented with aluminum. Since the electrode 150 is implemented with the component with these characteristics, the electrode 150 can operate as shown in FIG. 3.

In some implementations, the electrodes 150 can be provided as a pair of the electrodes 150 per the lamp 140, and a plurality of lamps 140 can be disposed in a row with each other.

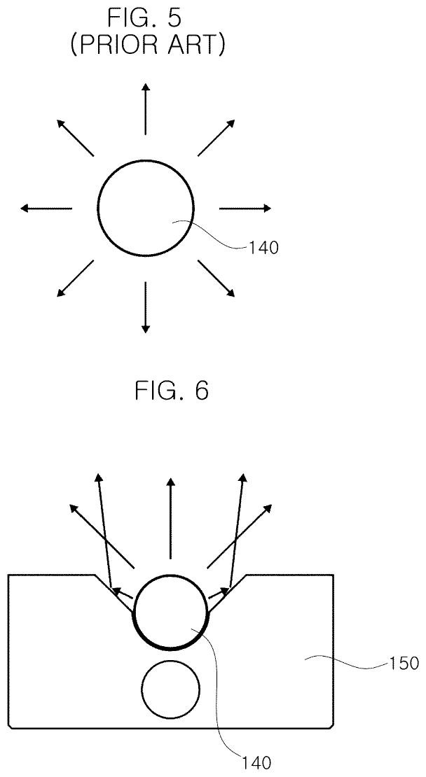

FIG. 5 is a diagram illustrating an operation of a conventional lamp, and FIG. 6 is a diagram illustrating a lamp in the UV lamp device according to the present disclosure.

As shown in FIG. 5, when the lamp 140 emits light, the light is radiated in all directions, which may be desirable in some cases. In some cases, when the light emitted from the lamp 140 should be emitted in one direction (for example, a vertically upward direction in FIG. 3), as shown in FIG. 5, when the light is emitted from the lamp 140, the light emitted in directions other than the direction in which the light should be emitted causes inefficiency.

In some examples, as shown in FIG. 6, since the lamp 140 is seated on the electrodes 150 and the electrodes 150 have the above-described characteristics, the light emitted from the lamp 140 that is emitted in a direction opposite to the direction in which the light should be emitted is reflected from the electrodes 150 and travels in the direction in which the light should be emitted.

In particular, the electrode 150 can have a shape shown in FIG. 6 in order to improve reflectivity for light incident on the electrode 150. That is, a lower end surface of the groove H of the electrode 150 has a semi-cylindrical outer surface, a depth of a lower end portion has a size that is less than or equal to a radius of the lamp 140, and since the groove H is in surface contact with the lamp 140, the lamp 140, which is usually a circular shape, can be stably seated.

In some examples, an upper end portion of the groove H of the electrode 150 has a shape that is inclined upward from the lower end portion and extends. For example, an inclination angle of the upper end surface can range from 30° to 60° with respect to a flat bottom surface of the electrode 150. In some examples, the inclination angle can be 45°±5° to provide reflection efficiency for uniformly reflecting the light in one direction.

In some implementations, the trigger 160 can be configured to cause the lamp 140 to output the light. Even in a situation in which a voltage is applied to the lamp 140, when a trigger does not occur around the lamp 140, emission of light from the lamp 140 may not progress. Thus, the trigger 160 is disposed adjacent to the lamp 140 and triggers the lamp 140. In some examples, the trigger 160 can include an electric circuit.

As shown in FIG. 4, the trigger 160 is disposed between the electrodes 150 to trigger emission of the UV light from the lamp 140. In some implementations, the trigger 160 can include a hollow cap and a coil disposed inside the cap. For example, the cap can have a cylindrical shape. The cap of the trigger 160 can have a shape in which both end portions of the cap are inserted into a pair of the electrodes 150, and according to the size and structure of the electrode 150, the cap can be formed between the electrodes 150 adjacent to a lower portion of the lamp 140 or between the electrodes 150 adjacent to a lateral side of the lamp 140. In some examples, trigger grooves 151 can be formed in the two electrodes 150, and the trigger 160 can be inserted between the trigger grooves 151 to maintain a spacing between the electrodes 150.

The coil in the trigger 160 is disposed inside the cap and is in contact with only one of the electrodes 150. Thus, the coil in the trigger 160 can generate a spark to apply stimulation or electrical stimulation by the light using the lamp 140. The lamp 140 can be triggered by the stimulation generated by the trigger 160 and can emit light with the UV wavelength range by receiving a high voltage.

The controller controls an operation of each component in the UV lamp device 100. For example, the controller can include an electric circuit, a processor, a computer, or a microprocessor. In some examples, the vehicle can include the controller.

In some examples, the controller can control each component in the UV lamp device 100 to perform the above-described operation.

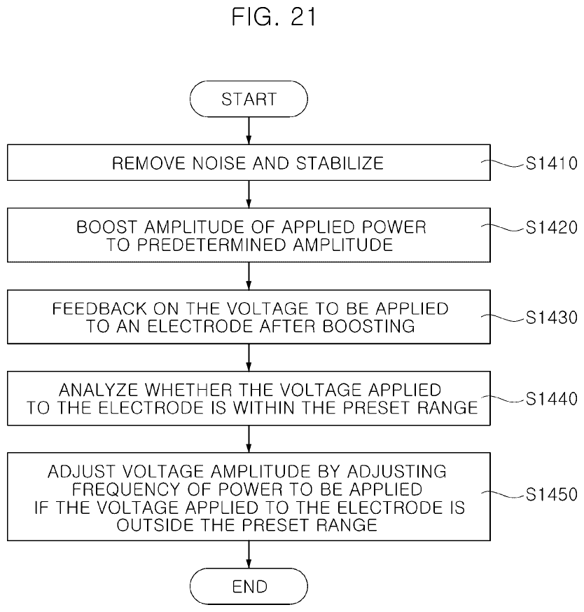

In some examples, the controller monitors the voltage applied to the trigger 160 to determine whether appropriate light is being output from the lamp 140. This process is shown in FIG. 21.

FIG. 21 is a flowchart illustrating a method of monitoring light output from a UV lamp device.

When power is supplied from the outside, the controller removes a noise introduced with the supplied power and stabilizes the supplied power (S1410).

The controller boosts a voltage (e.g., several kV) to an amplitude of the applied power to be applied to the electrodes 150 (S1420). The controller applies the voltage to the electrodes 150. The voltage is applied to the electrodes 150, the trigger 160 is triggered, and the lamp 140 emits light.

The controller receives feedback of a portion of the voltage applied to the electrodes 150 and analyzes an amplitude (S1430). The controller can use an isolated operational amplifier (OP AMP) in the process of receiving feedback of the portion of the voltage applied to the electrodes 150. As described above, the voltage applied to the electrodes 150 corresponds to a state of being significantly boosted. When the voltage is fed back directly to the controller, there is a risk that the controller can be damaged or adversely affected. In order to prevent the problem, the controller can use the isolated OP AMP to lower the voltage by a preset ratio during the process of receiving the feedback of the portion of the voltage.

The controller analyzes whether the voltage applied to the electrodes 150 is present within a preset range (S1440). When a frequency of the voltage applied to the electrodes 150 increases, an amplitude of the voltage decreases and in the opposite case, the amplitude of the voltage increases.

Using this point, when the voltage applied to the electrodes 150 is out of a preset range, the controller adjusts a frequency of power to be applied to adjust an amplitude of the voltage (S1450). When the voltage applied to the electrodes 150 does not reach a preset range, the controller decreases the frequency of the voltage applied to the electrodes 150 (or adjusts a duty ratio to decrease the frequency) to increase the amplitude of the voltage. In some examples, when the voltage applied to the electrodes 150 exceeds the preset range, the controller increases the frequency of the voltage applied to the electrodes 150 (or adjusts the duty ratio to increase the frequency) to decrease the amplitude of the voltage. The controller receives feedback of the portion of the voltage applied to the electrodes 150 and analyzes the amplitude of the voltage, thereby enabling a uniform voltage to be applied to the electrodes 150.

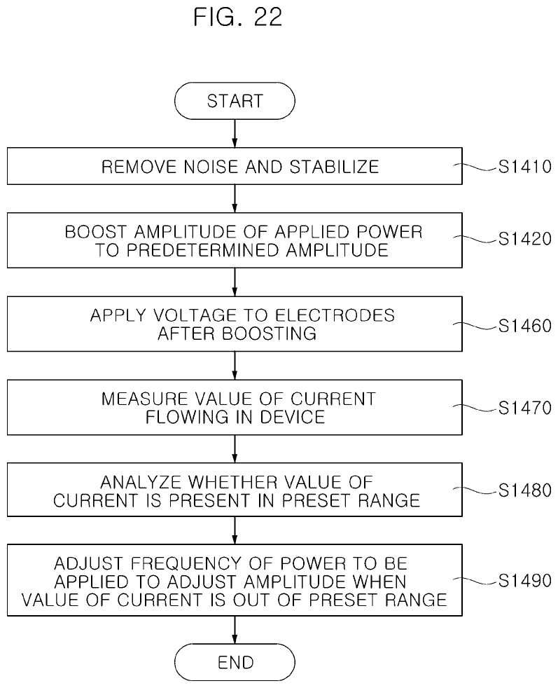

FIG. 22 is a flowchart illustrating a method of monitoring light output from a UV lamp device.

The processes of S1410 and S1420 are performed as the same as S1410 and S142 in FIG. 21.

The controller applies a voltage to the electrodes after the boosting (S1460).

Separately, the controller stabilizes the applied power and then measures a value of a current flowing in the UV lamp device 100 (S1470).

The controller analyzes whether a value of the current is present in a preset range (S1480). Even in a situation in which the lamp 140 is lighting stably, output power should be present in a preset range (3.6 W to 4.3 W). In some cases, even in a situation in which the lamp 140 is not lighting stably, the output power is out of the preset range (for example, 3 W). The controller calculates the output power of the lamp 140 using the value of the measured current and analyzes whether the calculated power satisfies the preset range.

When the value of the current is out of the preset range, the controller adjusts a frequency of power to be applied to adjust an amplitude of the voltage (S1490). As described above, the controller adjusts the frequency or a duty ratio of the voltage applied to the electrodes 150 on the basis of the analysis result.

In some examples, the UV lamp device 100 can further include the lamp fixing part. The lamp fixing part is disposed at a position for pressurizing the lamp 140 in the housing 120 to fix the lamp 140. For example, the lamp fixing part is implemented with an elastic component such as a spring and is disposed to pressurize the lamp 140 between the housing 120 or the cover 110 and the lamp 140. The lamp fixing part can be implemented with the above-described configuration to pressurize the lamp 140, thereby preventing damage concerns of the lamp 140 and fixing the lamp 140 not to be moved from its original position.

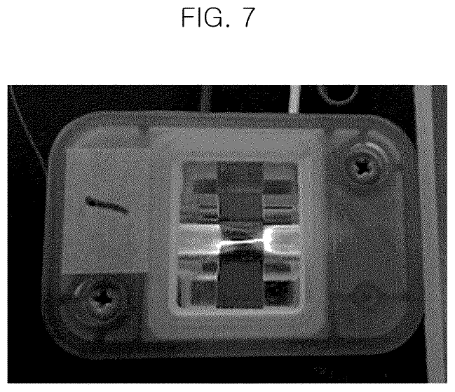

The UV lamp device 100 of the present disclosure can have the above-described structure and operate as described above, thereby having output characteristics as shown in FIGS. 7 to 9.

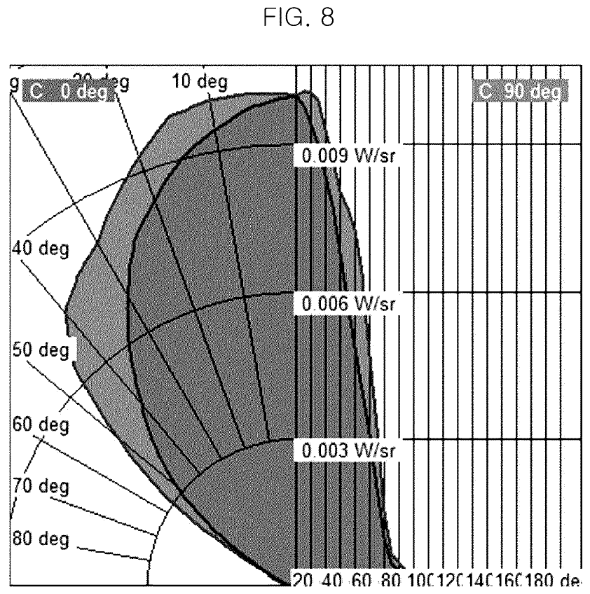

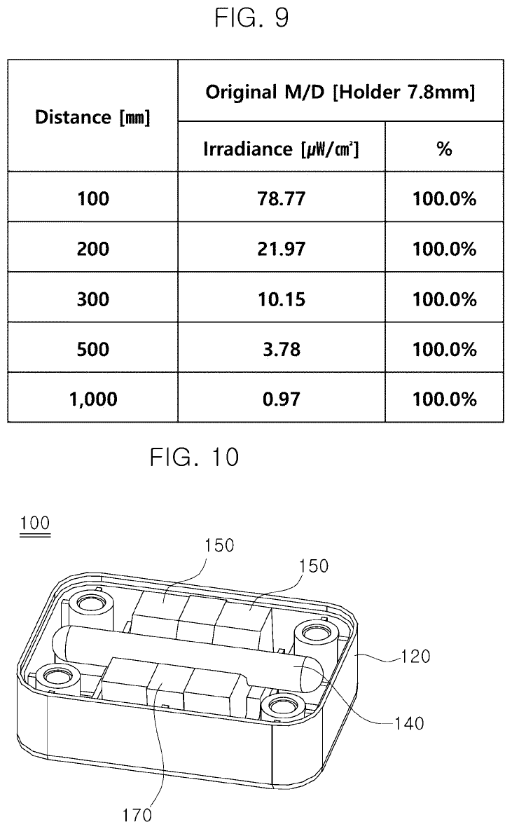

FIG. 8 is a diagram illustrating an output result of the UV lamp device, and FIG. 9 is a diagram illustrating output data of the UV lamp device.

As shown in FIG. 8, the UV lamp device 100 intensively outputs light in the UV wavelength range in a region where the lamp 140 is disposed on the electrodes 150.

In addition, as shown in FIG. 9, the UV lamp device 100 can have appropriate radiation range and radiation efficiency and output light in the UV wavelength range.

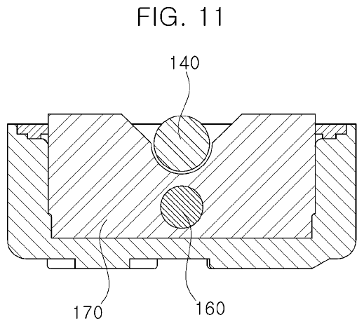

FIG. 10 is a diagram illustrating an additional configuration of the UV lamp device 100, and FIG. 11 is a diagram illustrating a side cross-sectional shape.

Referring to FIGS. 10 and 11, the UV lamp device 100 can include a reflector 170.

The reflector 170 can be implemented to have the same cross section as the electrode 150 or can be implemented to include a u-shaped groove for seating the lamp 140 like the electrodes 150. A shape feature of a lower end portion and an upper end portion of the groove can be identical to that of the above-described groove H of the electrode 150. The reflector 170 is implemented as described above and disposed between the two electrodes 150. In order to improve efficiency of the light to be output, in some examples, the electrodes 150 and the reflector 170 are disposed to be in close contact therewith.

In some examples, a hole corresponding to the trigger 160 can be formed.

The reflector 170 can be implemented with a material that is non-conductive and has excellent reflective characteristics in the UV wavelength range (more specifically, a UV-C wavelength range) and can be implemented with, for example, a Teflon material. When the reflector 170 is conductive, the two electrodes 150 can electrically conduct so that a problem of a short circuit can occur. In some examples, the reflector 170 can be implemented with a non-conductive material. A distance of the two electrodes 150 in the UV lamp device 100 is maintained by the cap of the trigger 160.

In some examples, where the reflector 170 is included, the distance of the two electrodes 150 in the UV lamp device 100 can be maintained stably by the cap of the trigger 160 as well as by the reflector 170.

In some implementations, where the reflector 170 is disposed between the two electrodes 150, as shown in FIG. 7, the light in the UV wavelength range can be emitted throughout the entire region of the lamp 140, including a region where the lamp 140 is disposed on the electrodes 150, rather than the light is intensively emitted to the region where the lamp 140 is disposed on the electrodes 150. In particular, since an output amount of the light is not reduced in the region where the lamp 140 is disposed on the electrodes 150, but rather the light is equally reflected and output in the remaining region, a light emission efficiency of the UV lamp device 100 can be significantly improved.

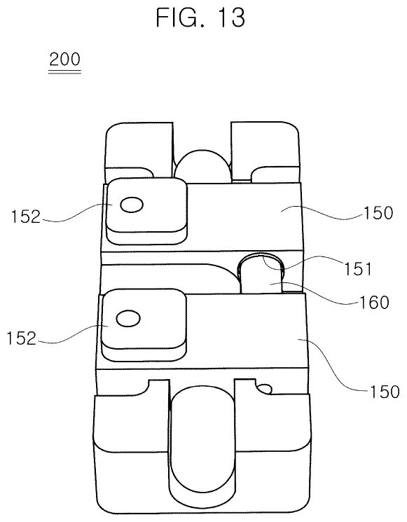

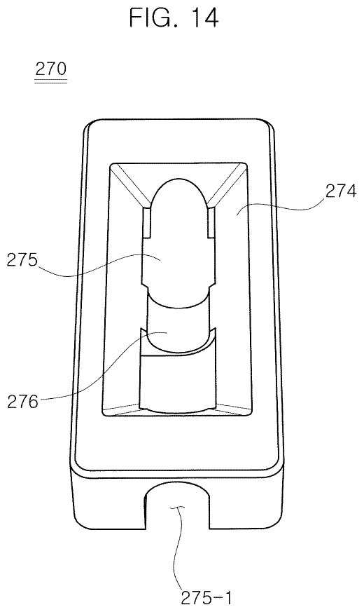

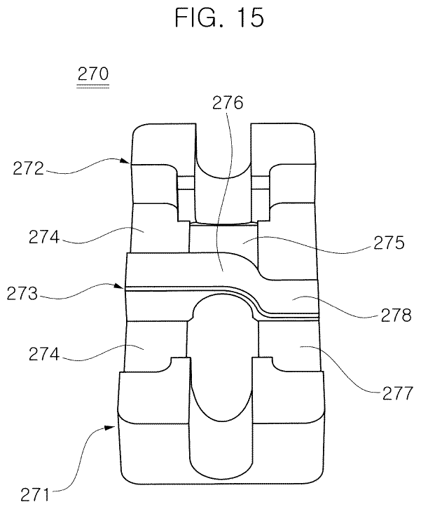

FIG. 12 is a front open perspective view illustrating an example of a UV lamp device, FIG. 13 is a rear open perspective view, FIG. 14 is a front perspective view illustrating a reflector of the UV lamp device of FIG. 12, and FIG. 15 is a rear perspective view.

Referring to FIGS. 12 to 15, while a UV lamp device 200 will be described, a reflector 270 will be mainly described, and other components that are the same as those of the UV lamp device 100 will be omitted.

While the reflector 170 of FIG. 10 is disposed between the electrodes 150, the reflector 270 seats a lamp 140 therein and reflects light emitted from all positions of the lamp 140 together with electrodes 150 in a direction in which the light should be emitted.

In some implementations, the reflector 270 includes a first body 271 formed between one electrode of the pair of electrodes 150 and the housing 120, a second body 272 formed between the other electrode of the pair of electrodes 150 and the housing 120, a third body 273 formed between the pair of electrodes 150, and an upper surface portion 274 connecting upper surfaces of the first body 271, the second body 272, and the third body 273.

A passing-through portion 275 through which the lamp 140 passes is formed in the first body 271 and the second body 272, the lamp 140 is inserted through an inlet portion 275-1, and a groove identical to the groove H of the lamp device 100 is formed in the third body 273 to form a lamp mount 276.

Therefore, the reflector 270 is formed to cover the entire space around the lamp 140, and the upper surface portion 274 forms a reflective surface by covering the upper surfaces of the electrodes 150.

That is, the upper surface portion 274 and the groove of the lamp mount 276 form the reflective surface.

In addition, a lower portion of the upper surface portion 274 becomes an electrode mount 277 on which the electrodes 150 are mounted, and one side of two sides based on the groove of the third body 273 is formed to be stepped compared to the other side so that the stepped side forms a trigger mount 278.

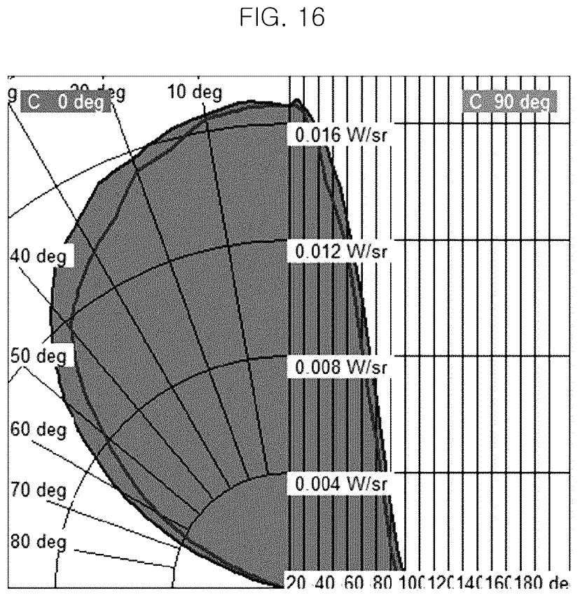

With this structure, the UV lamp device 200 can have output characteristics as shown in FIG. 16 due to higher reflection efficiency.

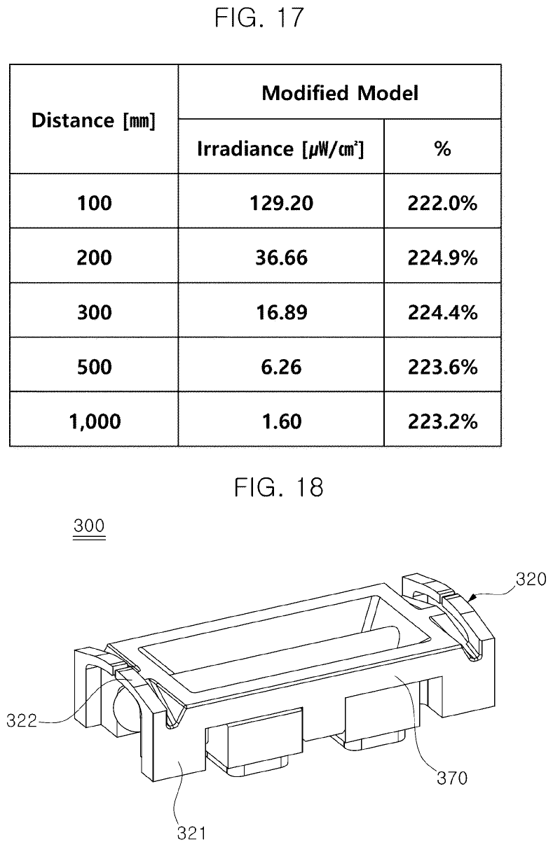

FIG. 17 is a diagram illustrating output data of the UV lamp device.

As shown in FIG. 17, the UV lamp device 200 can have appropriate radiation range and radiation efficiency and output light in the UV wavelength range. In particular, it can be confirmed that the UV lamp device 100 can output light in an emission range that is significantly increased compared to the UV lamp device 100, and the emission efficiency is also improved by a factor of three times or more compared to the UV lamp device 100. Since the reflector 270 and the electrodes 150 can reflect the light emitted from all positions of lamp 140, the emission range and emission efficiency can be significantly improved.

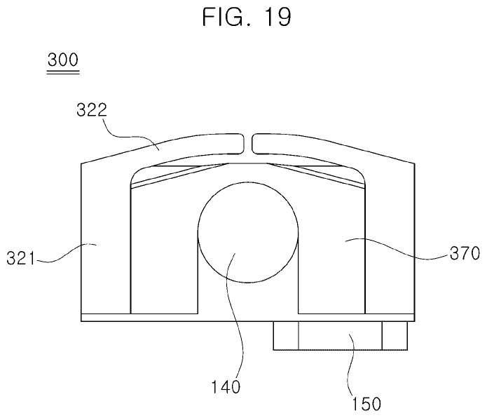

FIG. 18 is a diagram illustrating an example configuration of a reflector in a UV lamp device, and FIG. 19 is a diagram illustrating a side cross-sectional shape.

An ultraviolet lamp device 300 can include a reflector 370 that has a similar configuration as the reflector 270 in the ultraviolet lamp device 200 and additionally includes pressurizing parts 320 extending from the first body 271 and the second body 272 in a longitudinal direction.

The pressurizing part 320 extends from side surfaces of an end of a body and includes a pressurizing support 321 having a notch formed on an upper portion thereof and deformation portions 322 formed to extend from an upper end of the pressurizing support 321 to face each other.

The deformation portions 322 can have a shape of an upwardly curved arch and are in contact with the cover 110 when the cover 110 is coupled to the housing 120. Thus, the deformation portions 322 are in contact with the cover 110 and receive a force from the cover 110 in a direction that presses the deformation portions 322. When the deformation portions 322 receive a force in the corresponding direction, the force in the corresponding direction is directly transmitted to the lamp 140 through a body. Accordingly, the lamp 140 can be prevented from being separated from the body.

That is, since the pressurizing part 320 is included in the lamp device 300, the lamp 140 is structurally fixed even without including a separate lamp fixing part and is easily coupled and elastically supported when the cover 110 is coupled, and the pressurizing support 321 facilitates deformation of the deformation portions 322.

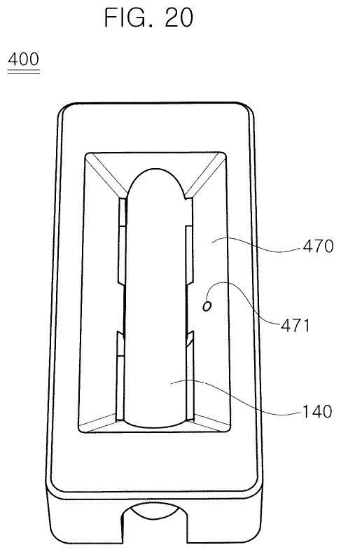

FIG. 20 is a diagram illustrating an example configuration of a UV lamp device.

A UV lamp device 400 can include a reflector 470 which is similar to the reflector 270 of the UV lamp device 200. The reflector 470 has a trigger pass-through hole 471 defined therein. For example, the trigger pass-through hole 471 is shown as being implemented in the reflector 470, but the present disclosure is not necessarily limited thereto. In some cases, trigger pass-through hole 471 can be implemented in the electrode 150.

A body in the reflector 470 is non-conductive, but can be implemented with a material that reflects light in the UV wavelength range. That is, the body of the reflector 470 can either transmit or reflect light in the remaining wavelength range. In some cases, when the body of the reflector 470 reflects the light in the remaining wavelength range, there is a probability that a trigger generated in the trigger 160 is not transmitted to the lamp 140.

To reduce the probability, the trigger pass-through hole 471 is implemented in the reflector 470 and/or the electrode 150. It is sufficient that the trigger pass-through hole 471 is implemented with an area enough for the trigger to pass through and reach the lamp 140. Since the trigger pass-through hole 471 is implemented, a trigger generated in the trigger 160 can be transmitted to the lamp 140 through the trigger pass-through hole 471.

As described above, the present disclosure can be easily applied to a narrow space such as a vehicle interior by increasing efficiency even in the same size and output through the electrode and reflector structures.

Test results of the structure to which the reflector of the present disclosure is applied and the existing structure are compared under the same conditions as follows.

TABLE 1

| | | | | No- | | 20 mm | Irra- |
|---|---|---|---|---|---|---|---|
| | | | | load | Lamp | irra- | diance |
| | Volt- | Cur- | Pow- | pow- | pow- | diance | improve- |
| | age | rent | er | er | er | (mW/ | ment |
| | (V) | (A) | (W) | (W) | (W) | cm²) | (%) |
| Before | 12 | 0.308 | 3.696 | 2.82 | 0.876 | 1.03 | 100 |
| improve- | | | | | | | |
| ment | | | | | | | |
| After | 12 | 0.313 | 3.756 | 2.80 | 0.948 | 2.3 | 223 |
| improve- | | | | | | | |
| ment | | | | | | | |

As described above, there is an improvement effect of approximately 223% after structural improvement as in the present disclosure.

According to the present disclosure, electrode and reflector structures can be improved to achieve maximum sterilization efficiency in a limited size.

Therefore, the present disclosure can be applied to narrow spaces in a vehicle, such as a console lower tray, a front tray, and a wireless charger of the vehicle and can increase a commercial value of the vehicle by cleaning a vehicle interior.

Although the implementations have been described with reference to a number of illustrative implementations of the technical spirit of the present implementation, it should be understood that numerous other modifications and implementations can be devised by those skilled in the art that will fall within the spirit and scope of the principles of the present implementation. The implementations disclosed herein, therefore, are not to be taken in a sense for limiting the technical concept of the present disclosure but for explanation thereof, and the range of the technical concept of the present implementation is not limited to these implementations. The scope of the implementations should be construed by the appended claims, along with the full range of equivalents to which such claims are entitled.

What is claimed is:

1. An ultraviolet (UV) lamp device for a vehicle, comprising:
a housing;
a cover that covers a surface of the housing;
a lamp disposed in the housing and configured to emit UV light; and
a pair of electrodes configured to apply a voltage to the lamp, the pair of electrodes being spaced apart from each other and supporting the lamp,
wherein the pair of electrodes define a groove that accommodates at least a portion of the lamp and supports a bottom surface of the lamp and a side surface of the lamp.

2. The UV lamp device of claim 1, wherein a depth of a lower end portion of the groove is less than or equal to a radius of the lamp, and
wherein an inner lower surface of the lower end portion of the groove has a semi-cylindrical outer surface corresponding to a shape of the lamp.

3. The UV lamp device of claim 2, wherein an upper end portion of the groove extends upward from the lower end portion of the groove and is inclined with respect to a bottom surface of the pair of electrodes.

4. The UV lamp device of claim 3, wherein a depth of the groove is less than or equal to a diameter of the lamp.

5. The UV lamp device of claim 3, wherein an inclination angle of the upper end portion of the groove ranges from 30° to 60° with respect to the bottom surface of the pair of electrodes.

6. The UV lamp device of claim 3, wherein an inclination angle of the upper end portion of the groove is 45°±5° with respect to the bottom surface of the pair of electrodes.

7. The UV lamp device of claim 2, further comprising:
a trigger disposed adjacent to the lamp and configured to cause the lamp to emit the UV light.

8. The UV lamp device of claim 7, wherein the trigger comprises a cap and a coil disposed inside the cap.

9. The UV lamp device of claim 8, wherein the pair of electrodes define trigger grooves that receive end portions of the trigger, respectively.

10. The UV lamp device of claim 2, further comprising:
a filter coupled to an open surface of the housing and configured to transmit a predetermined wavelength range of light.

11. The UV lamp device of claim 2, wherein the cover defines a pair of through-holes, and the pair of electrodes comprise terminals that pass through the pair of through-holes and are exposed to an outside of the cover.

12. The UV lamp device of claim 2, further comprising:
a reflector disposed between the pair of electrodes and configured to support the lamp.

13. The UV lamp device of claim 12, wherein the reflector is in contact with the pair of electrodes.

14. The UV lamp device of claim 12, wherein the reflector defines a groove that has a cross-sectional shape corresponding to the semi-cylindrical outer surface of the groove of the pair of electrodes.

15. The UV lamp device of claim 2, further comprising a reflector that includes:
a first body disposed between a first electrode of the pair of electrodes and the housing;
a second body disposed between a second electrode of the pair of electrodes and the housing; and
a third body disposed between the pair of electrodes.

16. The UV lamp device of claim 15, wherein the reflector further includes an upper surface portion that connects between upper surfaces of the first body and the third body and that connects between an upper surface of the second body and the upper surface of the third body.

17. The UV lamp device of claim 16, wherein:
the first body and the second body define passing-through portions through which the lamp passes; and
the third body defines a groove that has a cross-sectional shape corresponding to the semi-cylindrical outer surface of the groove of the pair of electrodes.

18. The UV lamp device of claim 16, wherein the third body has a first side and a second side with respect to the groove of the third body, the second side being stepped relative to the first side, and
wherein the UV lamp device further comprises a trigger disposed at the second side of the third body and configured to cause the lamp to emit the UV light.

19. The UV lamp device of claim 16, further comprising:
a pressurizing part that extends from at least one of the first body or the second body in a longitudinal direction of the lamp and is configured to be pressed by the cover.

20. The UV lamp device of claim 19, wherein the pressurizing part includes:
a pair of pressurizing supports that extend from a side surface of the first body or the second body in the longitudinal direction, each of the pair of pressurizing supports having a notch defined at an upper portion thereof; and
a pair of deformation portions that extend from upper ends of the pair of the pressurizing supports, respectively, and face each other, the pair of deformation portions being configured to be pressed by the cover.
